(19) United States
(12) Patent Application Publication
(10) Pub. No.: US 2009/0093030 A1
(43) Pub. Date: Apr. 9, 2009

(54) fadR KNOCK-OUT MICROORGANISM AND METHODS FOR PRODUCING L-THREONINE

(75) Inventors: Young Hoon Park, Gyeonggi-do (KR); Jin Ho Lee, Gyeonggi-do (KR); Byoung Choon Lee, Seoul (KR); Dae Cheol Kim, Gyeonggi-do (KR); Jae Yong Cho, Gyeonggi-do (KR)

Correspondence Address: ARENT FOX LLP, 1050 CONNECTICUT AVENUE, N.W., SUITE 400, WASHINGTON, DC, 20036, US

(73) Assignee: CJ Corporation

(21) Appl. No.: 11/381,453

(22) Filed: May 3, 2006

Related U.S. Application Data
(62) Division of application No. 10/683,381, filed on Oct. 10, 2003, now abandoned.

(30) Foreign Application Priority Data
Oct. 11, 2002 (KR) 10-2002-0062103

(51) Int. Cl. C12P 13/08; C12N 1/21; C12N 15/70

(52) U.S. Cl. 435/115; 435/252.33; 435/471; 435/320.1

(57) ABSTRACT

The present invention relates to an L-threonine-producing chromosomal fadR gene knock-out microorganism. The present invention further relates to a method for producing L-threonine using a fadR knock-out microorganism. Mutated microorganisms of the present invention are capable of increased L-threonine production.

This application claims priority to Korean Patent Application No. 10-2002-0062103 filed Oct. 11, 2002 the contents of which are incorporated herein in their entirety by reference.

### BACKGROUND OF THE INVENTION

1. Field of the Invention

The present invention relates to L-threonine-producing fadR knock-out microorganisms and a process for producing L-threonine by fermentation of these microorganisms. A non-limiting working example provided is a mutated microorganism in which the fadR gene present on the chromosome of the microorganism has been knocked out using a site-specific Cre/loxP homologous recombination system. The mutant microorganism displays a phenotype in which the expression of the aceBAK operon is derepressed during L-threonine biosynthesis.

2. Description of the Art

L-threonine is an essential amino acid and is widely used as a feed and food additive. It is also used as a pharmaceutical agent and as a synthetic precursor for some drugs. It has been produced by fermentation with mutants of the genus Escherichia, Coryneform bacteria, Seratia and Providencia. For example, Japanese Patent Publication No. S56-10037 discloses a method for producing L-threonine using a strain belonging to the genus Escherichia which has a nutritional requirement for diaminopimeric acid and methionine and is resistant to feedback inhibition of the threonine biosynthetic pathway by threonine. Japanese Patent Application Laid-open No. S58-224684 discloses a method for producing L-threonine using a strain belonging to the genus Brevibacterium which is resistant to S-(2-aminoethyl)-L-cysteine and α-amino-β-hydroxy valeric acid and has a nutritional requirement for L-isoleucine and L-lysine. Korean Patent Application Laid-open No. 87-8022 discloses a method for producing L-threonine using a methionine-requiring, α-amino-β-hydroxy valeric acid-resistant strain belonging to the genus Escherichia which has an additional resistance to at least one substance selected from the group consisting of rifampicin, lysine, methionine, aspartic acid, and homoserine and has a reduced ability to decompose L-threonine. Japanese Patent Application Laid-open No. H2-219582 discloses a method for producing L-threonine using a strain belonging to the genus Providencia which is resistant to α-amino-α-hydroxy valeric acid, L-ethionine, thioisoleucine, oxythiamine, and sulfaguanidine and has a nutritional requirement for L-leucine and also a leaky requirement for L-isoleucine.

However, the foregoing methods fail to provide high yield L-threonine production or require expensive nutritional substances such as diaminopimiric acid and isoleucine. For example, the use of diaminopimeric acid-requiring strains increases L-threonine production costs due to the need for diaminopimeric acid in the growth media. Similarly, the cost of isoleucine limits the use of the foregoing isoleucine-requiring strains to produce L-threonine inexpensively.

An L-threonine-producing strain of Escherichia coli which is resistant to α-aminobutyric acid and L-methionine, L-threonine and L-lysine analogues and has a nutritional requirement for methionine and a leaky requirement for isoleucine was developed previously. This strain produced more L-threonine by fermentation than prior strains. This strain and a method for producing L-threonine using said strain are described in Korean Patent Publication No. 92-8365, published Sep. 26, 1992.

In addition, a number of patents disclose methods for preparing genetically engineered bacterial strains capable of producing threonine. Such patents include U.S. Pat. Nos. 4,278,765, 6,132,999, 4,321,325 and 5,175,107 which relate to genetically engineered bacterial strains harboring a hybrid vector containing genes required for synthesis of threonine. Further, U.S. Pat. Nos. 6,297,031 and 6,165,756 disclose recombinant bacterial strains having inactivated threonine dehydrogenase activity.

Oxaloacetate, which is a synthetic precursor of threonine, may be formed by the tricarboxylic acid cycle (TCA). Alternatively, oxaloacetate may be formed from phosphoenolpyruvate by phosphoenolpyruvate carboxylase, which is encoded by the ppc gene. Microorganisms that have the thrA gene encoding the enzyme aspartokinase I/homoserine dehydrogenase may convert oxaloacetate to the non-essential amino acid aspartate. Aspartate is an intermediate for the synthesis of lysine, methionine, threonine. Of these amino acids, the synthesis of threonine further requires the thrB and thrC genes, which respectively encode homoserine kinase and threonine synthase.

The present inventors have continued to develop strains with the ability to produce higher L-threonine yields and have focused on the fadR gene, which encodes a transcription factor, FadR, that coordinately regulates both fatty acid synthesis and degradation. Without being limited to any particular model, the basis for this approach is that fatty acid metabolism affects the level of oxaloacetate, which is a synthetic precursor of threonine, in the cell.

More specifically, FadR negatively regulates fatty acid catabolism by repressing transcription of the fatty acid transport and β-oxidation genes fadL, fadD, fadE, fadF, fadG, fadBA, and fadH (Raman N et al., 1997, JBC 272:30645-30650; DiRusso C C et al., 1992, JBC 267:8685-8691). On the other hand, FadR activates fatty acid biosynthesis by transcriptionally activating the fadA and fadB genes, which are required for unsaturated fatty acid synthesis (J. E. Cronan Jr. and D. Laporte, E. coli and Salmonella, 1996, vol 1, pp 211-214). FadR also negatively regulates the glyoxylate shunt by transcriptionally activating IclR, a repressor of the aceBAK operon (Gui L et al., 1996, J. Bacteriol. 178:4704-4709). The aceBAK operon encodes the glyoxylate shunt enzymes isocitrate lyase, malate synthase, and isocitrate dehydrogenase kinase/phosphatase. Expression of the aceBAK operon is induced during growth on acetate or fatty acids (Farmer W R et al., 1997, Applied Environ. Microbiol. 63:3205-3210).

The glyoxylate shunt pathway is used by microorganisms to metabolize acetate or long chain fatty acids as a source of carbon. It diverts intermediates away from the tricarboxylic acid (TCA) cycle when the organisms are exposed to low oxygen conditions. Acetyl CoA from acetate or fatty acids is combined with oxaloacetate to form citrate which is then converted to isocitrate. However, in microorganisms grown on acetate or fatty acids, further progress through the TCA cycle is blocked because the activity of isocitrate dehydrogenase (IDH) is attenuated by phosphorylation by IDH kinase/phosphorylase. Instead, isocitrate is converted to glyoxylate by isocitrate lyase and subsequently to malate by malate synthase. Malate may then be converted to oxaloacetate.

The citric acid cycle is subject to the biosynthesis of oxaloacetate from pyruvate through eight (8) pathways and, during the process, intermediates are converted into other metabolic intermediates including carbon dioxide. If the expression of aceBAK operon decreases, carbon flow through the glyoxylate cycle is inhibited, thereby increasing formation of other metabolic intermediates through the citric acid cycle, which results in decreased production of L-threonine against identical amounts of carbon source.

The present inventors have succeeded in isolating a knock-out fadR mutant microorganism with the ability to produce a high concentration of L-threonine in which aceBAK expression is increased. In addition, the present inventors have used the Cre/loxP site-specific recombination system to knock out the fadR gene.

### SUMMARY OF INVENTION

The present invention provides fadR knock-out microorganisms capable of producing L-threonine at higher levels than previously achievable and methods of producing L-threonine using such microorganisms.

In some embodiments, the present invention provides an L-threonine-producing microorganism in which the fadR gene present on the chromosome of the microorganism has been knocked out.

Some embodiments of the present invention provide a method for producing fadR knock-out microorganisms comprising constructing a knock-out cassette of the fadR gene or DNA fragment thereof, introducing the construct into the reference L-threonine-producing microorganism such that recombination occurs between the foreign construct and the fadR gene present on the chromosome of the microorganism, and selecting the mutated microorganism in which the fadR gene has been knocked out.

In some embodiments of the invention, a process is provided for producing L-threonine by fermentation which comprises culturing the microorganism in which the fadR gene present on the chromosome of the microorganism has been knocked out, and purifying L-threonine from the culture.

The present invention also provides a knock-out cassette of fadR gene or DNA fragment thereof constructed by inserting an antibiotic marker flanked by loxP site at both ends into the fadR gene, and the loxP site is used to remove the antibiotic marker from the chromosome of integrated mutants by expressing cre gene encoding loxP site-specific recombinase.

### BRIEF DESCRIPTION OF THE DRAWINGS

FIG. 1 depicts a construction of recombinant plasmid pT7Blue/fadR of the present invention.

FIG. 2 depicts a construction of DNA fragment ΔfadR::loxpcat from recombinant plasmid pT7fadR::loxpcat.

FIG. 3 shows a chromosomal recombination of the fadR gene of the present invention by Southern blot analyses.

### DETAILED DESCRIPTION OF THE INVENTION

In accordance with the present invention, microorganisms capable of producing elevated levels of threonine may be produced by knocking out the chromosomal fadR gene. In some non-limiting embodiments, the invention provides an exemplary microorganism as well as methods of producing such a knock-out microorganism by homologous recombination. Without being limited to any particular mechanism of action, the expression of the aceBAK operon may not be fully repressed in fadR knock-out microorganisms of the invention. In some non-limiting embodiments, the invention provides methods of producing L-threonine by culturing a microorganism of the invention under conditions that permit L-threonine production.

Some prokaryotic and eukaryotic microorganisms with the ability to produce L-threonine can be used for the present invention. Representative examples of appropriate microorganisms include, but are not limited to, mutated strains belonging to the genus Escherichia, Coryneform bacteria, Seratia and Providencia which have been used in the art to produce L-threonine by fermentation. In some preferred embodiments of the present invention, the microorganisms include, but are not limited to, an L-threonine-producing strain of Escherichia coli which is resistant to α-aminobutyric acid, analogues of L-methionine, analogues of L-threonine, and analogues of L-lysine and has a nutritional requirement for methionine and a leaky requirement for isoleucine. In some preferred embodiments of the present invention, the microorganisms comprise, in addition to intrinsic genes coding for phosphoenolpyruvate carboxylase (ppc) and enzymes included in the threonine operon, at least one copy of a gene selected from the group consisting of an exogenic ppc gene, a thrA gene, a thrB gene, and a thrC gene. These exogenous genes may be inserted in the chromosomal threonine operon.

The chromosomal fadR gene may be knocked out by an appropriate knock-out mutation technique known to those skilled in the art. “Knock-out mutation” as used herein refers to an engineered disruption of native chromosomal DNA, typically within a protein coding region, such that a foreign piece of DNA is inserted within the native sequence. The foreign DNA may contain a gene of interest and/or encode a marker. The marker may be selected from the group consisting of a dominant selectable marker (e.g. antibiotic resistance), a histochemical tag, a fluorescent marker, and combinations thereof. The gene of interest may encode a protein. According to the invention, a knock-out mutation within a protein coding region prevents expression of the wild-type protein and usually leads to loss of the function provided by the protein.

In some embodiments of the invention, if an antibiotic marker is integrated into chromosome during a first knock-out procedure, the marker can not be used again to knock out another gene. So, it may be desirable or necessary to remove the marker from chromosome.

The knock-out procedure can be carried out by mixing a knock-out cassette with a culture of a L-threonine-producing microorganism competent for DNA uptake.

While the microorganism is naturally transformable it is preferred that cells can be rendered competent for DNA uptake by any suitable method (See e.g. LeBlanc et. al., 1992, Plasmid 28:130-145; Pozzi et al., 1996, J. Bacteriol. 178: 6087-6090). A “knockout cassette” refers to a fragment of native chromosomal DNA having a foreign DNA piece that may provide a selectable marker. In one embodiment “knock-out mutation cassettes” are created by interrupting a fragment of genomic DNA with a foreign piece of DNA, and replacing the wild-type chromosomal copy of the sequence with the knock-out cassette. In this embodiment, the knock-out protocol involves cloning a foreign DNA piece into a target DNA such that “tails” comprising the target site DNA remain at the 5′ and 3′ ends of the knock-out cassette. The tails should be at least 50 base pairs and preferably greater than 200 to 500 base pairs for efficient recombination and/or gene conversion. For convenience, the foreign DNA cloned into the target DNA also provides a selectable marker, for example, an antibiotic resistance gene. Where the target DNA is disrupted with an antibiotic resistance gene, selection of transformants is carried out on agar plates containing suitable levels of an appropriate antibiotic. Following transformation, a fraction of cells that have taken up the knockout cassette will have undergone homologous recombination or gene conversion across the genomic DNA tails of the cassette, resulting in replacement of the wild-type genomic sequence by the knock-out cassette. Knock-out recombination events are easily confirmed by, for example, Southern blot hybridization, or more conveniently by PCR.

In some non-limiting embodiments, preparation of a knockout mutation of the present invention comprises the following procedures. Genomic DNA is isolated from a strain that is capable of producing L-threonine and PCR is performed to amplify the genomic fadR gene. The PCR product obtained containing the fadR gene is cloned into a suitable plasmid or other vector. The cloned insert may be positively identified as fadR or a fadR fragment by any means known in the art including, without limitation, PCR, Southern blotting, DNA sequencing, and combinations thereof.

This recombinant vector is introduced by transduction into a host cell such as E. coli. After the transformant is grown in the culture medium, the recombinant vector having fadR genes is extracted. An antibiotic resistant gene fragment is then inserted into the fadR gene of the extracted recombinant vector to form a knock-out recombinant construct. The recombinant insert may be positively identified as having the antibiotic marker inserted in the desired location by any means known in the art including, without limitation, PCR, Southern blotting, DNA sequencing, and combinations thereof.

This recombinant vector is introduced by transformation into a host cell and the host cell is cultivated in a suitable culture medium. Then, the propagated recombinant vector is isolated from the resultant transformant, and the knockout cassette fragment of the fadR gene is obtained by suitable restriction enzyme digestion(s). Thereafter, this fragment is introduced into a host that is capable of producing L-threonine by a conventional technique such as electroporation under conditions that permit homologous recombination. Microorganisms in which a recombination event have occurred are selected in antibiotic-containing media. The microorganisms so selected may be isolated by conventional techniques and DNA may be extracted and sequenced to confirm the site of recombination. In some non-limiting preferred embodiments, DNA sequencing may be performed to identify mutant microorganisms in which the wild-type fadR gene has been disrupted. In some non-limiting preferred embodiments, PCR using particular primers may be performed to identify mutant microorganisms in which the wild-type fadR gene has been disrupted. In some non-limiting preferred embodiments, Southern blotting may be performed to identify mutant microorganisms in which the wild-type fadR gene has been disrupted.

Skilled artisans will recognize that the knockout cassettes and the DNA segments of this invention or fragments thereof can be generated by general cloning methods. PCR amplification methods using oligonucleotide primers targeted to any suitable region of any of the sequences disclosed herein are preferred. Methods for PCR amplification are widely known in the art. See e.g. PCR Protocols: A Guide to Method and Application, Ed M. Innis et al., Academic Press (1990) or U.S. Pat. No. 4,889,818, which are hereby incorporated by reference. The PCR comprises genomic DNA, suitable enzymes, primers, and buffers, and is conveniently carried out in a DNA Thermal Cycler (Perkin Elmer Cetus, Norwalk, Conn. USA). A positive PCR result is determined by, for example, detecting an appropriately-sized DNA fragment following agarose gel electrophoresis.

The fadR gene may be knocked out by any means known in the art. Recombination may be achieved or facilitated using any recombination system known in the art including, without limitation, the Cre/lox recombination system, the Flp/FRT recombination system, the bacteriophage P22 recombination system, the bacteriophage integrase/att recombination system, and the XerCD recombination system.

While an L-threonine-producing microorganism of the invention may be produced by knocking out the fadR gene, the invention provides other means of inactivating the fadR gene or gene product including, without limitation, chemical mutagenesis, irradiation mutagenesis (e.g. by exposure to ultra violet light and/or X-rays), PCR mutagenesis, viral, transposon, or other insertional mutagenesis, deletion of from one fadR nucleotide to the entire fadR gene, frameshift mutation, point mutation, epigenetic modification, chromosomal translocation, recombination, addition of cis- or trans-acting regulatory elements, and RNA interference. These techniques are known to one of ordinary skill in the art as illustrated, for example, in Maniatis, Fritsch & Sambrook, Molecular Cloning: A Laboratory Manual (2001) and Ausubel, Current Protocols in Molecular Biology (1998).

In some preferred embodiments, the present invention uses, as an L-threonine-producing microorganisms, L-threonine-producing strains of Escherichia coli which are resistant to L-methionine, L-threonine and L-lysine analogues and α-aminobutyric acid and have a nutritional requirement for methionine and a leaky requirement for isoleucine.

L-methionine analogues include, but are not limited to, D,L-ethionine, norleucine, α-methylmethionine and L-methionine-D,L-sulfoximine. L-threonine analogues include, but are not limited to, α-amino-β-hydroxy valeric acid and D,L-threonine hydroxamate. L-lysine analogues include, but are not limited to, S-(2-aminoethyl)-L-cysteine and δ-methyl-L-lysine.

In some especially preferred embodiments, the present invention uses, as an L-threonine-producing microorganism, a novel mutant of Escherichia coli, which has a chromosomal fadR gene knock-out. This mutant strain is capable of producing L-threonine in higher yield than the parent strain, Accession No. KCCM 10236. This mutant was prepared by first providing knockout cassette ΔfadR::loxpcat. The knockout cassette ΔfadR::loxpcat is cleaved from a recombinant plasmid construct pT7fadR::loxpcat which is derived from recombinant plasmid construct pT7blue/fadR. Second, an Escherichia coli strain that is resistant to L-methionine, L-threonine and L-lysine analogues and α-aminobutyric acid and has a nutritional requirement for methionine and a leaky requirement for isoleucine, namely Escherichia coli Accession No. KCCM 10236, was provided. Finally, the Escherichia coli Accession No. KCCM 10236 was transformed with the knockout cassette ΔfadR::loxpcat by electroporation. The novel mutant was designated as Escherichia coli FTR1201 and was deposited under the Budapest Treaty to the Korean Culture Center of Microorganisms whose address is Hongje-dong, Seodaemun-gu, Seoul 120-749, on Sep. 13, 2002 and assigned Accession No. KCCM-10422.

Escherichia coli Accession No. KCCM 10236 was mutated from Escherichia coli Accession No. KCCM TF4076 (Accession No. KFCC 10718) which requires methionine and has resistance to L-threonine analogues (for example, α-amino-α-hydroxy valeric acid), L-lysine analogues (for example, S-(2-aminoethyl-L-cysteine), L-isoleucine analogues (for example, α-aminobutyric acid), methionine analogues (for example, ethionine) and the like.

Escherichia coli Accession No. KCCM TF4076 is described in Korean Patent Publication No. 92-8365 which is incorporated herein in its entirety by reference. Escherichia coli Accession No. KCCM 10236 differs from Escherichia coli Accession No. KCCM TF4076 in that it possesses two phosphoenolpyruvate carboxylase (ppc) genes and two L-threonine (thr) operons. The ppc gene and thr operon originated from the chromosomes of Escherichia coli Accession No. KCCM TF4076 were amplified by the polymerase chain reaction and were additionally integrated into the chromosomes of Escherichia coli Accession No. KCCM TF4076 to generate Escherichia coli Accession No. KCCM 10236.

The ppc gene catalyzes the formation of oxaloacetate from phosphoenolpyruvate. As such, phosphoenolpyruvate is a precursor of oxaloacetate which is an intermediate for the threonine biosynthetic pathway. Escherichia coli Accession No. KCCM 10236 is, therefore, capable of expressing higher levels of the enzymes necessary for threonine biosynthesis, thereby enabling an increase in L-threonine production.

Genomic DNA is extracted from Escherichia coli Accession No. KCCM 10236. PCR is performed using the genomic DNA as template to amplify the fadR gene. The PCR products are loaded onto agarose gel and subjected to electrophoresis. The fadR gene or DNA fragment thereof isolated thus is cloned into an appropriate vector. A DNA fragment of the gene conferring resistance to an antibiotic is inserted within the fadR gene region of the constructed recombinant vector to induce knockout of the fadR gene.

The knocked-out fadR gene cassette including the DNA fragment for antibiotic resistance is isolated from the resulting recombinant vector. The isolated knocked-out fadR gene cassette is transformed into L-threonine-producing microorganism such as Escherichia coli Accession No. KCCM 10236 by conventional methods such as electroporation to produce a mutated strain with the ability to highly produce L-threonine.

As used in the preparation of the knocked-out fadR gene or fragments thereof, the term “vector” refers to a nucleic acid molecule capable of carrying the foreign fadR gene or DNA fragment thereof to which it has been linked and introducing the fadR gene or DNA fragment into host cells. Examples of the vector are plasmids, cosmids, viruses and bacteriophages originated from natural sources or recombinantly synthesized.

The production of L-threonine by using the mutated L-threonine-producing microorganisms of the present invention, in which the fadR gene present on the chromosome of the microorganism has been knocked out, may be carried out by an ordinary method for culturing host cells such as bacteria and yeast. In some embodiments of the invention, any synthetic media and/or natural media may be used as the culture media for L-threonine production as long as it appropriately contains carbon sources, nitrogen sources, inorganic substances and trace amounts of nutrients which the strain requires.

Examples of the carbon sources include carbohydrates such as glucose, fructose, lactose, molasses, cellulose hydrolysate, crude sugar hydrolysate and starch hydrolysate, organic acids such as pyruvic acid, acetic acid, fumaric acid, malic acid and lactic acid, and alcohols such as glycerin and ethanol. Examples of the nitrogen sources include ammonia, various inorganic salts (such as ammonium chloride, ammonium sulfate, ammonium acetate and ammonium phosphate), ammonium salts of organic acids, amines, peptone, meat extract, corn steep liquor, casein hydrolysate, soybean cake hydrolysate, various fermented cells and digested matters thereof.

Examples of the inorganic substances include potassium dihydrogenphosphate, dipotassium hydrogenphosphate, magnesium phosphate, magnesium sulfate, magnesium chloride, sodium chloride, ferrous sulfate, manganese sulfate, copper sulfate, calcium chloride and calcium carbonate.

Culturing is carried out under aerobic conditions, for example, by shaking culture or spinner culture under aeration. The culturing temperature is in the range of 20 to 40° C., preferably 28 to 37° C. The pH of the medium is in the range of pH 5 to 9, preferably around neutrality. The pH of the medium may be adjusted by adding, for example, calcium carbonate, an organic or inorganic acid, an alkali solution, ammonia, or a pH buffer.

Usually, L-threonine is formed and accumulated in the culture by 1 to 7 days of culturing. An optional process for cultivation includes, for example, any of continuous operation, semi-continuous operation, and batch operation.

After the culturing is completed, precipitates such as cells are removed from the culture, and L-threonine can be recovered from the culture by means of ion exchange chromatography, concentration, salting-out, etc. alone or in combination. For example, the recovery of L-threonine from the culture broth may be carried out by the following method. The culture broth from which the cells are removed may be adjusted to pH 2 with hydrochloric acid. Then the broth solution may be passed through a strongly acidic ion exchange resin, and the adsorbant may be eluted by dilute aqueous ammonia. Ammonia may be evaporated and then the resulting solution may be condensed. Alcohol may be added to the concentrate, and then, crystals formed under cooling may be collected, and then L-threonine may be obtained.

According to the invention, the increase in L-threonine production by the knock-out mutants of the invention over the corresponding wild-type strains is about 1%, more preferably about 3%, more preferably about 8%, and more preferably about 9%.

The following non-limiting examples further describe the present invention. However, the examples are offered solely for illustration and, therefore, are not to be construed as limiting the scope or subject matter of the instant invention. The contents of all documents cited throughout this application are expressly incorporated herein by reference in their entirety. Citation of these documents should not be construed as an admission that any document(s) is prior art against the claims.

### EXAMPLE 1

### Construction of Recombinant Plasmid and Knock-Out of fadR Gene

Genomic DNA was extracted from L-threonine-producing Escherichia coli strain Accession No. KCCM 10236 by using the QIAGEN Genomic-tip System. The DNA fragment (about 0.8 kb) including ORF (open reading frame) of fadR gene was amplified by PCR using the extracted genomic DNA as a template and a pair of oligonucleotides 5′-TCG CGG AAG AGT ACA TTA TTG-3′ (forward primer; SEQ ID NO: 1) and 5′-ATC GGC GCA AAG AAG TCC-3′ (reverse primer; SEQ ID NO:2). PCR was performed by 30 cycles, each consisting of denaturation for 30 seconds at 94° C., annealing for 30 seconds at 55° C. and extension for 60 seconds at 72° C. in order.

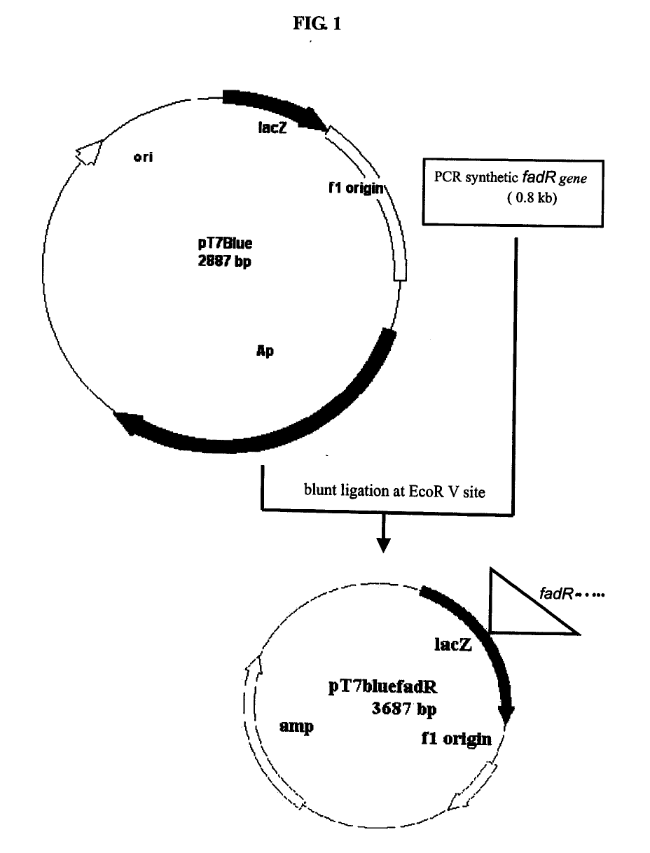

The PCR product was loaded onto 1.0% agarose gel and subjected to electrophoresis. The 0.8 kb fadR gene band was cut out of the gel and eluted. The resulting fadR gene was ligated to EcoRV site of cloning vector pT7Blue (Novagen Inc., USA) overnight at the temperature of 16° C. to construct the recombinant plasmid pT7Blue/fadR (see FIG. 1). The resulting plasmid construct was transformed into Escherichia coli NM522. The transformed strain was plated on solid media containing 50 mg/L of carbenicillin and was cultured overnight at a temperature of 37° C.

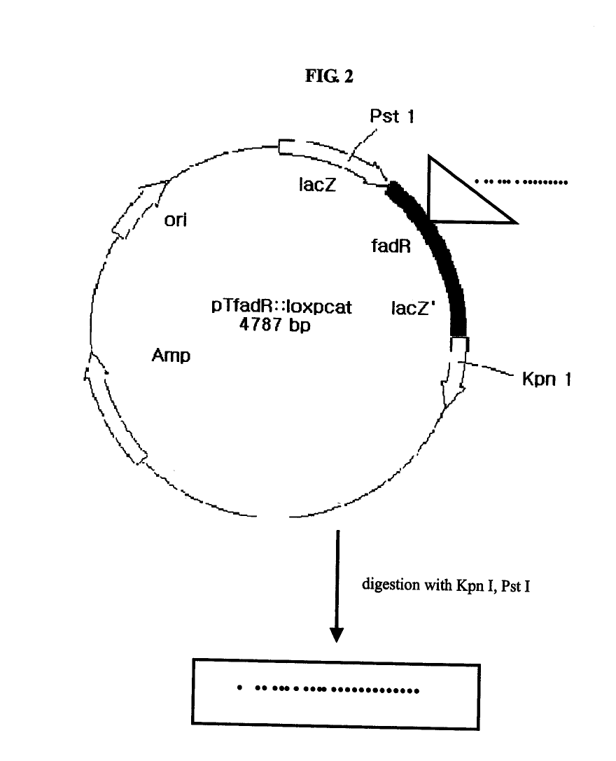

The colonies formed were picked up with a platinum loop and inoculated into 3 ml of liquid media containing carbenicillin. After overnight culturing, plasmid DNAs were extracted from the culture using QIAGEN Mini Prep Kit. The plasmid DNA extract was digested with the restriction enzyme SacII and confirmed the cloning of fadR gene. The confirmed plasmid pT7Blue/fadR was cleaved with SacII and DNA fragments formed thus were run over 0.8% agarose gel. A band of about 3.8 kb was cut out of the gel and eluted. The 3.8 kb DNA fragment was blunt-ended by the treatment of Klenow enzyme. The resulting DNA fragment was blunt-end ligated with about 1.1 kb fragment of the gene for chloramphenicol resistance including loxP region, which was obtained by digesting plasmid ploxpcat2 (Gosset et al, “A family of removable cassettes designed to obtain Ab-resistance-free genomic modifications of E. coli” Gene 247:255-264) with HincII restriction enzyme, to construct about 5.0 kb recombinant plasmid pT7ΔfadR::loxpcat (see FIG. 2).

Escherichia coli NM522 was transformed with the recombinant plasmid pT7ΔfadR::loxpcat. The resulting transformant was streaked out onto a solid medium plate containing 50 mg/L of carbenicillin and 15 mg/L of chloramphenicol and cultured overnight at 32° C. The colonies formed were picked up with a platinum loop and inoculated into 3 ml of liquid media containing carbenicillin and chloramphenicol.

After overnight culturing, plasmid DNAs were extracted using QIAGEN Mini Prep Kit. The plasmid DNA extract was digested with KpnI and PstI restriction enzymes and DNA fragments formed thus were run over 0.7% agarose gel. About 2.2 kb band was cut out of the gel and eluted (see FIG. 2). The resulting DNA fragment, about 2.2 kb, ΔfadR::loxpcat was transformed into Escherichia coli Accession No. KCCM 10236 by electroporation and the transformed Escherichia coli Accession No. KCCM 10236 was streaked out onto an agar medium containing chloramphenicol. The selected colonies were tested for their production of L-threonine in flask cultures.

### EXAMPLE 2

L-threonine Production in Erlenmeyer Flask by Selected Strains

Thirty colonies selected in Example 1 were cultured in an Erlenmeyer flask containing the threonine titration medium given in Table 1 below, and L-threonine production was compared.

TABLE 1

| Ingredients | Concentration (per liter) | |
|---|---|---|
| Glucose | 70 | g |
| Ammonium sulfate | 28 | g |
| KH₂PO₄ | 1.0 | g |
| MgSO₄•7H₂O | 0.5 | g |
| FeSO₄•7H₂O | 5 | mg |
| MnSO₄•8H₂O | 5 | mg |
| Calcium carbonate | 30 | g |
| L-methionine | 0.15 | g |
| Yeast extract | 2 | g |

pH 7.0

Each colony was cultured on LB agar medium in a shaking incubator at 32° C.

One platinum loop of the culture was inoculated into 25 ml of the titration medium and shaking cultured at 32° C. and 250 rpm for 48 hours. The titration results are given in Table 2 below.

TABLE 2

| L-threonine | 23 g/L | 24 g/L | 25 g/L | 26 g/L |
|---|---|---|---|---|
| Strain number | 1 | 23 | 5 | 1 |

It can be seen from the results that the transformants of the present invention produce an average of about 24-25 g/L L-threonine. Therefore, transformed Escherichia coli Accession No. KCCM 10236 of the present invention in which fadR gene has been knocked out is improved over the prototype strain Accession No. KCCM 10236 which produces 23 g/L L-threonine. In addition, it was observed that the present transformed microorganisms increase the output of L-threonine in fermentation media up to about 8% in comparison to the prototype strain. One transformant among them was designated as Escherichia coli FTR1201 (Accession No. KCCM-10422).

### EXAMPLE 3

### Confirmation of fadR Gene Knock-Out by Southern Blotting

A Southern blot was performed to confirm whether fadR gene has been rendered knocked out in the strains selected in Example 2. The prototype strain Accession No. KCCM 10236 and the present strains FTR1201 were cultured overnight on 3 ml of a liquid medium containing chloramphenicol. Genomic DNA was extracted from the culture using QIAGEN Genomic Kit 20.

The extracted genomic DNA was cleaved overnight with the restriction enzyme XmnI. The resulting DNA fragments were size fractionated by electrophoresis on an 0.7% agarose gel. After completion of electrophoresis, the DNA molecules in the agarose gel were transferred onto a nylon membrane (YOUNG Sci. Biodyne B Membrane) using a capillary transfer method (Molecular Cloning, vol. 1. pp 6.31-638). The membrane was dried and then the DNA molecules were immobilized on the dry membrane by UV irradiation (120 mJ/cm², SpectroLinker™).

The membrane was treated with a prehybridization solution I (Roche #1093657) at 55° C. for 2 hours. After addition of the denatured DNA probe, hybridization was performed overnight in 55° C. oven (BAMBINO 230300).

The denatured DNA probe used was prepared as follows. The isolated plasmid ploxpcat2 was digested with HincII restriction enzyme using QIAGEN Kit to obtain about 1.1 kb DNA fragment of the gene for chloramphenicol resistance including loxP region. The resulting DNA fragment was heated in water at 100° C. for 5 minutes and soon cooled on ice for 5 minutes to give single-stranded DNA molecules which was DIG-labeled at 37° C. using a DIG labeling and detection kit (Roche #1093657) to produce the DIG-LDP-labeled DNA probe.

After completion of the hybridization, the DNA molecules nonspecifically hybridized on the membrane were removed using the wash solutions I and II (Roche #1093657). The membrane was masked with the prehybridization buffer solution 2 (Roche #1093657) at a room temperature for 30 minutes and reacted with anti-DIG antibody, which specifically binds to DIG-UTP, at a room temperature for 30 minutes.

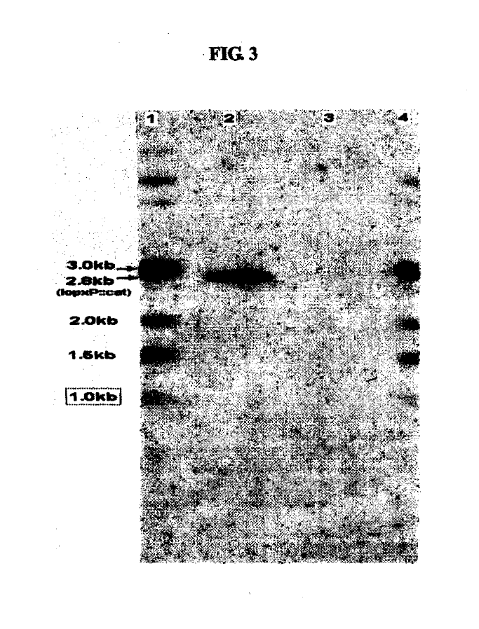

The anti-DIG antibody nonspecifically bound on the membrane was removed with the wash solution 111 (Roche #1093657) and the membrane was chromatized with the above label and detection kit (Roche #1093657) so as to show the band. The images were scanned using FLA-5000 Image System (FUJIFLIM). The results are given in FIG. 3. The prototype strain Accession No. KCCM 10236 (lane 3) showed no band because it did not possess the gene for chloramphenicol resistance. In contrast, the present strain FTR1201 (Accession No. KCCM 10422) (lane 2) showed about 2.8 kb band as expected. It is understood that the band consists of 1.7 kb portion of fadR gene and about 1.1 kb DNA fragment of the gene for chloramphenicol resistance.

### EXAMPLE 4

### Production of L-threonine in a Fermenter

The production of L-threonine by the present strain FTR1201 (Accession No. KCCM-10422) in a 5 L fermenter was compared with that by the prototype strain Accession No. KCCM 10236. The contents of the inoculum medium are shown in Table 3 below.

TABLE 3

| Ingredients | Concentration (per liter) | |
|---|---|---|
| Glucose | 50 | g |
| KH₂PO₄ | 4 | g |
| (NH₄)₂SO₄ | 6 | g |
| Yeast extract | 3 | g |
| MgSO₄•7H₂O | 2 | g |
| L-methionine | 1 | g |
| FeSO₄•7H₂O | 40 | mg |
| MnSO₄•8H₂O | 10 | mg |
| CaCl₂•2H₂O | 40 | mg |
| CoCl₂•6H₂O | 4 | mg |
| H₃BO₃ | 5 | mg |
| Na₂MoO₄•2H₂O | 2 | mg |
| ZnSO₄•7H₂O | 2 | mg |

pH 7.0

An LB medium supplemented with 10 g/L of glucose and 0.1 g/L of L-methionine was used for the seed culture. The initial inoculum volume in the fermenter was adjusted to from 3% to 5% of the initial culture volume. Glucose was added six times each at its depletion point. After each addition, the concentration of glucose was 5%. At the time of adding glucose, 1% by weight of mono-potassium phosphate (KH₂PO₄) was also added. The initial and final culture volumes were 1.5 L and 3.0 L, respectively, and the concentration of total glucose added until completion of fermentation was 250 g/L. Fermentation was performed with aeration of 0.5 vvm and stirring of 1,000 rpm at a temperature of 32° C. for 90 hours. The pH was maintained at 7.0 by an automatic inlet providing from 25% to 28% of ammonium gas.

The results are given in Table 4 below.

TABLE 4

| Strain | L-threonine Conc. (g/L) | Yield (%) |
|---|---|---|
| Prototype (KCCM 10236) | 93.5 | 37.4 |
| FTR1201 (KCCM 10422) | 102 | 41 |

The prototype strain 10236 produced 93.5 g/L of L-threonine as 37.4% yield to the glucose consumed. By contrast, the FTR1201 strain of the invention produced 102 g/L of L-threonine as 41% yield to the glucose consumed and thus increased about 9% of L-threonine yield in comparison to the prototype strain.

Thus, the L-threonine-producing mutated microorganism of the present invention in which fadR gene has been knocked out enhances the production of L-threonine.

1-11. (canceled)

12. A process for producing L-threonine comprising:
cultivating an L-threonine-producing microorganism whose chromosomal fadR gene has been knocked out under conditions that permit production of L-threonine, wherein L-threonine is produced.

13. The process of claim 12, wherein said cultivation comprises:
inoculating a culture media with said microorganism; and
incubating said inoculated culture media for at least about 1 day to about 7 days at from about 28° C. to about 37° C. with substantially constant shaking,
wherein a fermented media comprising L-threonine is produced.

14. The process of claim 13 further comprising isolating L-threonine from the fermented culture media.

15. The process of claim 18, wherein the concentration of L-threonine in the fermented media is at least about 1.0% higher than the concentration of L-threonine in the fermented media resulting from cultivation of a parent strain of Escherichia coli under substantially the same conditions.

16. The process of claim 15, wherein the concentration of L-threonine in the fermented media is at least about 3.0% higher than the concentration of L-threonine in the parent Escherichia coli fermented media.

17. (canceled)

18. The process of claim 12 wherein the microorganism is Escherichia coli.

19. The process of claim 18 wherein the microorganism is Escherichia coli FTR 1201 strain KCCM-10422.
